(19) United States
(12) Patent Application Publication
(10) Pub. No.: US 2015/0226717 A1
(43) Pub. Date: Aug. 13, 2015

(54) AUTOMATED ACCEPTANCE TESTING FOR BULK GAS DELIVERY

(71) Applicant: Salvatore F. Calandra, III, Nazareth, PA (US)

(72) Inventor: Salvatore F. Calandra, III, Nazareth, PA (US)

(21) Appl. No.: 14/615,726

(22) Filed: Feb. 6, 2015

Related U.S. Application Data
(60) Provisional application No. 61/938,715, filed on Feb. 12, 2014.

(51) Int. Cl. G01N 33/00

(52) U.S. Cl. CPC G01N 33/004

(57) ABSTRACT

A method (10) of testing the quality of a gas for discharge includes using a gas supplier for transporting a tank of gas to a customer storage facility and for testing the quality of the gas with customer analytical equipment at the storage facility for determining whether or not the gas is of quality standard sufficient to be released to the customer storage facility.

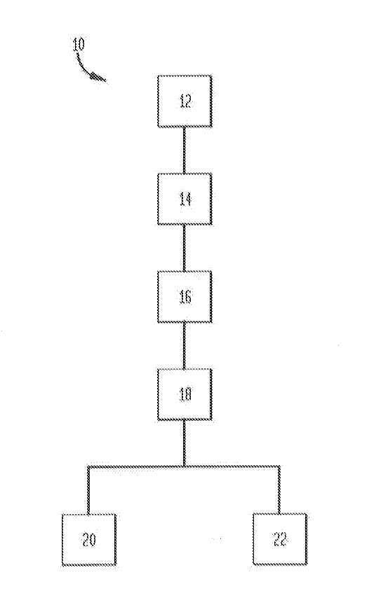

### BACKGROUND OF THE INVENTION

The present apparatus and method are related to testing of bulk gases, such as for example CO₂, for subsequent use in for example food, beverage, and electronics applications.

CO₂ use in industry is frequently transported in large tanker trucks, railcars, or in cylinders to be discharged at a processing plant or distribution facility. Depending upon the end use for the CO₂ product, certain testing of the CO₂ may be employed by the purchaser in order to insure quality assurance of the bulk and gas product for its intended use. For example, the required purity of the CO₂ used in metallurgical applications may be different than the purity necessary for use in food and beverage applications. Therefore, the quality of the CO₂ to be used can be different for these different industries and applications. Of course, testing and monitoring of the product at the point of delivery is typically the customer's responsibility and requires labor by plant personnel which can increase the amount of time necessary in order to determine whether the gas product meets quality assurance standards and can be discharged, or if the product fails the testing and will be rejected for use at the plant. Quality assurance/control and/or testing personnel are usually highly skilled in related protocols and analysis.

For certain deliveries of the bulk gas, a driver of the trailer carrying the bulk product may be delayed at the facility awaiting plant analytical personnel to arrive at the delivery point to test the gas product. This delay in time transfers along the distribution chain to increase uncertainty in the amount of time a transport trailer will be in position at the facility and of course, when it will be permitted to leave the facility after discharging product, should the product meet quality assurance standards. “Time is money” for bulk gas deliveries is certainly an accurate statement, and any apparatus or method employed which would reduce the amount of time necessary to test the hulk gas product and accurately schedule delivery of same will benefit the delivery system and supply chain,

A typical incoming quality control process used, for example, at carbonated soft drink production plants involves the bottling plant's quality control personnel performing two key functions prior to permitting gas to be discharged from delivery trailers: (i) verify trailer security seals on the gas transporters trailer, and (ii) perform testing of the CO₂ product using the semi-automated systems installed at the bottling plant.

In practice, due to a variety of factors (e.g. production monitoring) it is frequently a challenge for the bottler to make quality control personnel immediately available in a timely fashion during the CO₂ delivery process. This is because such personnel are usually involved in other aspects of plant operation remote from the bulk delivery and test site. Typically, upon the transport arriving at the bottling plant, the quality control department is contacted to meet the transport driver at the CO₂ tank station, Due to existing in-plant demands, in many cases the quality control personnel are not immediately available and it often takes 15-30 minutes on average for such personnel to become available and meet the transport driver. There are known cases where the driver has waited at least 2-4 hours for quality control personnel to become available. This is an unacceptable situation for both plant personnel and the transport driver.

Verifying the trailer seals (in (i) above) is a matter of matching the seal numbers on the trailer to the seal numbers recorded on the driver's paperwork. This is a relatively easy and quickly accomplished task, which can be performed by less skilled individuals other than the bother's analytical team, such as for example security or gate personnel.

Performing the quality testing (in (ii) above) on the CO₂ product is completed via an interface that requires a proficient, knowledgeable skill level to perform. Nevertheless, waiting for the bother's necessary testing personnel to arrive on site to perform the test is unnecessarily time consuming.

### BRIEF DESCRIPTION OF THE DRAWINGS

For a more complete understanding of the present embodiments, reference may be had to the following description taken in conjunction with the drawing FIGURE, of which:

The FIGURE shows a flow chart of the present embodiments to use the gas supplier to operate the test of the gas product delivered to the customer's tank with the customer's analytical equipment to determine gas quality.

### DETAILED DESCRIPTION OF THE INVENTION

The present embodiments provide a method (10) of testing the quality of a gas for discharge, including using a gas supplier for transporting a tank of gas to a customer storage facility (12) and for testing the quality of the gas (14) with customer analytical equipment at the storage facility without use of facility personnel.

The method may further include automating the testing for the gas supplier to determine the quality level of the gas (16).

The method may further include determining one of pass and fail of the testing for automatically determining whether the gas will be discharged to the storage facility (18).

The method may further include discharging the gas to the storage facility if said gas is at a minimum determined quality.

The method may also include retaining the gas in the tank if said gas is not at a minimum determined quality.

The gas may be CO₂.

The gas supplier may be a delivery truck,

Using CO₂ gas as an example only, the present embodiments provide an apparatus and method which automates the incoming quality control (QC) testing process for bulk CO₂ delivery by employing the gas delivery or transport driver (the “driver”) of the supplier to conduct a testing process to verify that the CO₂ conforms to the quality specification without facility personnel having to be present, or used to initiate, analyze and complete the testing process. A driver with minimal training is used to initiate the testing process to verify the CO₂ product quality being delivered via an automated interface. The interface communicates with the customer plant receiving analytical system for a determination if the gas may be discharged from the truck to the facility's gas storage tank. If the automated test fails, the driver is prevented from discharging the gas. The automated interface ensures that only CO₂ gas of a given quality is discharged. The driver cannot mistakenly discharge gas having an unacceptable quality level. Therefore, CO₂ gas which is not at the minimum desired quality level cannot be discharged. The interface is provided with the necessary interlocks to prevent the transport driver from discharging CO₂ unless same has successfully passed the facility's automated QC testing. Facility personnel do not therefore have to be involved in the quality testing.

The present embodiments obviate the need for the customer's quality assurance (QA) personnel to expend time and test each delivery of the bulk CO₂ for quality. The transport driver does not have to wait for QA personnel of the facility to become available and therefore, QA personnel can attend to other tasks at the facility. Further, because the present embodiments do not require the presence of QA personnel, it is possible to now deliver the CO₂ product regardless of customer lab personnel availability. This will enhance the reliability of supply to the bottling plant and permit a more efficient scheduling of deliveries.

There is therefore provided a method shown generally at 10 of testing the quality of a gas for discharge, consisting of transporting a bulk quantity of gas to a storage facility (12); employing a driver of the transporting of the bulk gas for testing a quality of the gas with the storage facility equipment (14); automating the testing to determine the quality of the gas (16); and determining one of pass and fail of the testing for determining whether or not the gas will be discharged to the storage facility (18). The gas may be for example CO₂.

If the quality of the gas is acceptable then the gas can be discharged by the driver to the storage facility (20). If the quality level of the gas is not acceptable, discharge will be denied (22) and the gas will be retained in the tank aboard the delivery truck so that the storage facility gas is not tainted.

In effect, the gas supplier (transport driver) is obtaining quality testing using the customer's (storage facility) analytical testing equipment, without the ability to discharge the gas product to the customer's storage facility if the product does not at least meet or exceed the customer's quality standard or specification requirements for the gas.

The present embodiments can substantially reduce CO₂ gas delivery time by at least 15 minutes to as much as 1½ hours.

The present embodiments can be used with any delivery platform for intermodal transport, e.g. truck, rail car, barge, etc.

It will be understood that the embodiments described herein are merely exemplary, and that one skilled in the art may make variations and modifications without departing from the spirit and scope of the invention, All such variations and modifications are intended to be included within the scope of the invention as described and claimed herein. Further, all embodiments disclosed are not necessarily in the alternative, as various embodiments of the invention may be combined to provide the desired result.

What is claimed is:

1. A method (10) of testing the quality of a gas for discharge, comprising
using a gas supplier for transporting a tank of gas to a customer storage facility (12) and for testing the quality of the gas (14) with customer analytical equipment at the storage facility.

2. The method of claim 1, further comprising automating the testing to determine the quality level of the gas (16).

3. The method of claim 1, further comprising determining one of pass and fail of the testing for automatically determining whether the gas will be discharged to the storage facility (18).

4. The method of claim 3, further comprising discharging the gas to the storage facility if said gas is at a minimum determined quality.

5. The method of claim 3, further comprising retaining the gas in the tank if said gas is not at a minimum determined quality.

6. The method of claim 1, wherein the gas is CO₂.

7. The method of claim 1, wherein the gas supplier is a delivery truck.
